(19) United States
(12) Patent Application Publication
(10) Pub. No.: US 2011/0200648 A1
(43) Pub. Date: Aug. 18, 2011

(54) Method and kit for cleansing underarms

(76) Inventor: Neil J. McCurry, Belmont, NH (US)

(21) Appl. No.: 12/658,875

(22) Filed: Feb. 17, 2010

(51) Int. Cl. A61K 8/02; A61K 8/86; A61Q 15/00; B65D 83/08

(52) U.S. Cl. 424/401; 424/65; 221/33; 221/45; 221/46

(57) ABSTRACT

A method and kit for cleansing one's underarms. The method includes the steps of applying a shaving cream, or other cleansing solution containing laureth-23 and sodium lauryl sulfate, to the underarm, waiting a sufficient amount of time, and removing the shaving cream from the underarm.

### FIELD OF THE INVENTION

The present invention relates to human body cleansing, and, in particular, to underarm cleansing.

### BACKGROUND

Body odor is a problem experienced by everyone at certain points in their lives. In particular, many people suffer from malodorous underarms, as the underarms are a particularly susceptible part of the body to have odor.

The underarm, or axilla, is proximal to an apocrine sweat gland. Apocrine sweat glands are coiled tubular glands that produce a viscous, cloudy, and potentially odorous secretion. The presence of bacteria growing in the axilla causes the odor, and that bacteria will multiply rapidly in the presence of the secretion from the sweat gland.

This odor can be difficult to counteract even with regular washing. This can be particularly difficult for men whose body odors tend to be more pungent than women's. It is also a particular challenge for adolescents who may be experiencing underarm malodor for the first time. Common soaps, even those identified as “deodorant” soaps or body washes, are not always effective at eliminating odors and preventing their reoccurrence.

As underarm odor may return after bathing, one may find oneself away from one's home when the underarm odor returns. In such situations, it may be difficult to sufficiently refresh the underarms without having a shower to use. Even if a shower is available to use, there may not be time for a shower, and/or the person with underarm odor may not want the hassle of taking an entire shower, which involves completely undressing, getting wet, bathing, drying, and completely redressing.

Therefore there is a need for a method of cleansing the underarm such that malodors are eliminated or reduced that may be performed quickly and in any location.

### SUMMARY OF THE INVENTION

The present invention includes a method and kit for cleansing the underarm. In a preferred embodiment, the method of the present invention includes the steps of applying a shaving cream containing laureth-23 and sodium lauryl sulfate, to the underarm, waiting a sufficient amount of time, and removing the shaving cream from the underarm.

The first step is applying the shaving cream to the underarm. Specifically, the first step is applying the shaving cream to dirty and/or malodorous underarms. In a preferred embodiment, the shaving cream is the shaving cream sold under the registered trademark BARBASOL BEARD BUSTER, by Perio, Inc, of Dublin, Ohio, but other shaving creams containing laureth-23 and sodium lauryl sulfate may be substituted to achieve similar results. Laureth-23 and sodium lauryl sulfate are compounds having detergent, surfactant, and deodorant properties. Not all shaving creams containing these ingredients are preferred, however. Some shaving creams sold under the registered trademark GILLETTE, by Gillette Company of Boston, Mass., for example, contain these ingredients and adequately cleanse the underarm, but irritate the skin during use and, consequently, are not preferred.

In another preferred embodiment of the method, the shaving cream is replaced by a cleansing solution containing laureth-23 and sodium lauryl sulfate usually included in shaving cream, but not all of the foaming agents of shaving cream so that the solution has a more liquid character than shaving cream. A preferred cleansing solution, for example, is a solution containing laureth-23, sodium lauryl sulfate, and fragrance, which are ingredients of the shaving cream sold under the registered trademark BARBASOL BEARD BUSTER, but not other ingredients for emulsifying the solution. In this way, when the cleansing solution is a solution containing these surfactants and detergents of shaving cream, but not all of its emulsifiers and propellants, the cleansing solution is essentially fluid and does not foam to the extent that shaving cream does.

In some embodiments, applying the shaving cream entails distributing the shaving cream into the hand and then wiping it on the underarm with the hand, or applying the shaving cream directly onto the underarm. In other embodiments, applying the shaving cream is accomplished by means of a brush. This brush may be attached to the shaving cream dispenser or may be a separate implement to be used in conjunction with the shaving cream dispenser. In any of these embodiments, applying the shaving cream preferably also entails spreading the shaving cream around the underarm and massaging the shaving cream into the underarm skin and/or hair.

The second step is waiting a sufficient amount of time for the ingredients of the shaving cream to counteract the malodor. A sufficient amount of time may be only the few seconds it may take to apply the shaving cream to the underarm, but may be up to two minutes. However, the preferred amount of time is thirty to sixty seconds. While waiting, the performer of the method of the present invention preferably continues to massage the shaving cream into the underarm.

The third step is removing the shaving cream from the underarm while leaving underarm hair substantially intact. In the preferred embodiment, this step entails wiping excess shaving cream from the underarm with a hand, washcloth, towelette, or other wipe. In other embodiments, this step entails providing sufficient water to the underarm area such that the shaving cream is rinsed away. In some embodiments, this step entails both wiping the shaving cream away and rinsing it away with water.

When the cleansing solution is a fluid solution containing some shaving cream ingredients, it is preferred that the solution's application be by a towelette impregnated with the solution. In other embodiments, the solution may be first applied to a cotton ball, cotton pad, towel, or other implement, and applied from there to the underarm. In the preferred method, however, the step of applying the solution is achieved by wiping the impregnated towelette over the underarm. Preferably, the performer of the method then massages the underarm with the towelette. The step of removing the solution is preferably achieved by allowing the solution to evaporate until the underarm is dry but may involve wiping the underarm with a dry towelette or one impregnated with water.

A preferred embodiment of the kit of the present invention is a towelette dispenser and disposable towelettes. In this embodiment the towelettes are preferably impregnated with a fluid solution that contains the surfactant and detergent ingredients laureth-23 and sodium lauryl sulfate usually included in shaving cream. A preferred cleansing solution, for example, is a solution containing laureth-23, sodium lauryl sulfate, and fragrance.

In another preferred embodiment, the towelette dispenser also dispenses towelettes, which may be dry or impregnated with water. In this embodiment, the towelette dispenser is partitioned and includes a barrier between the cleansing towelettes impregnated with cleansing solution and the removal towelettes so that the two are not in contact.

In another preferred embodiment, the towelette dispenser is a plastic or metallic wrapper and the cleansing solution impregnated towelettes are individually wrapped for ease of transport. In some such embodiments, the towelettes are two-sided with one side impregnated with cleansing solution and one side either dry or impregnated with water.

In another preferred embodiment, the kit includes a shaving cream dispenser and shaving cream and removal towelettes, which may or may not be impregnated with water. In some embodiments, the removal towelette dispenser and shaving cream dispenser are separate, but attached. In some embodiments, the removal towelette dispenser and shaving cream dispenser are combined into one dispenser, and that dispenser is partitioned and includes a barrier so that the removal towelettes and the shaving cream are not in contact.

Finally, it is noted that, although pronoun references herein are masculine, it is to be understood that the method and kit of the present invention may be used by women as well as men.

Therefore it is an aspect of the current invention to provide a method for cleansing underarms that eliminates odors therefrom.

It is a further aspect of the invention to provide a method for cleansing underarms that may be performed without the use of a source of water.

It is a further aspect of the invention to provide an underarm cleanser that may be packaged with an integral cleansing brush.

It is a further aspect of the invention to provide an underarm cleanser that may be impregnated within disposable towelettes.

It is a further aspect of the invention to provide an underarm cleansing kit that includes an underarm cleanser and a towelette for removing the cleanser.

These aspects of the present invention are not meant to be exclusive and other features, aspects, and advantages of the present invention will be readily apparent to those of ordinary skill in the art when read in conjunction with the following description, claims, and accompanying drawings.

### BRIEF DESCRIPTION OF THE DRAWINGS

FIG. 1 is a block diagram showing the steps of the method of the present invention.

FIG. 2A is a side view of a prior art basic shaving cream dispensing canister.

FIG. 2B is a side view of a prior art shaving cream dispensing canister with an extended nozzle.

FIG. 2C is a side view of a prior art shaving cream dispensing canister with a brush head nozzle.

FIG. 3 is a perspective view of a kit of the present invention that includes towelettes impregnated with cleansing solution.

FIG. 4A is a perspective view of a kit of the present invention that includes towelettes impregnated with cleansing solution and towelettes impregnated with water.

FIG. 4B is a side cutaway view of the kit of FIG. 4A.

FIG. 5A is top-down view of a preferred towelette dispenser.

FIG. 5B is a cross-section of a two-sided towelette.

FIG. 6A is a perspective view of a kit of the present invention that includes a water-impregnated towelette dispenser attached to a shaving cream dispenser.

FIG. 6B is a perspective view of one embodiment of a kit of the present invention that includes a dispenser for water-impregnated towelettes and shaving cream.

FIG. 6C is a side view of an alternate embodiment of a kit of the present invention that includes a dispenser for water-impregnated towelettes and shaving cream.

### DETAILED DESCRIPTION

The method of the present invention was discovered by the inventor when he needed to quickly cleanse his underarms and, being out of soap, used shaving cream instead. The inventor found that the shaving cream provided excellent results, which caused him to begin to experiment with the use of different shaving creams and soaps to determine which was most effective. This testing resulted in him concluding that the shaving cream sold under the registered trademark BARBASOL BEARD BUSTER, by Perio, Inc, of Dublin, Ohio was the most effective at eliminating underarm odors and was, in fact, superior to so called “deodorant soaps” and body wash soaps at this task. Further, he found that there was no need to rinse the shaving cream with water but, rather, he could simply wipe it off of his underarm, which caused the inventor to develop the kit of the present invention. Upon further experimentation, the inventor discovered that only those shaving creams that contain laureth-23 and sodium lauryl sulfate, such as that sold under the registered trademark BARBASOL BEARD BUSTER, by Perio, Inc, of Dublin, Ohio, gave the “squeaky clean” feeling he had initially discovered. In addition, he discovered that, although certain shaving cream products sold under the registered trademark GILLETTE, by Gillette Company of Boston, Mass. contain laureth-23 and sodium lauryl sulfate and provided the “squeaky clean” feeling, they were not desirable for this purpose because they irritated the skin.

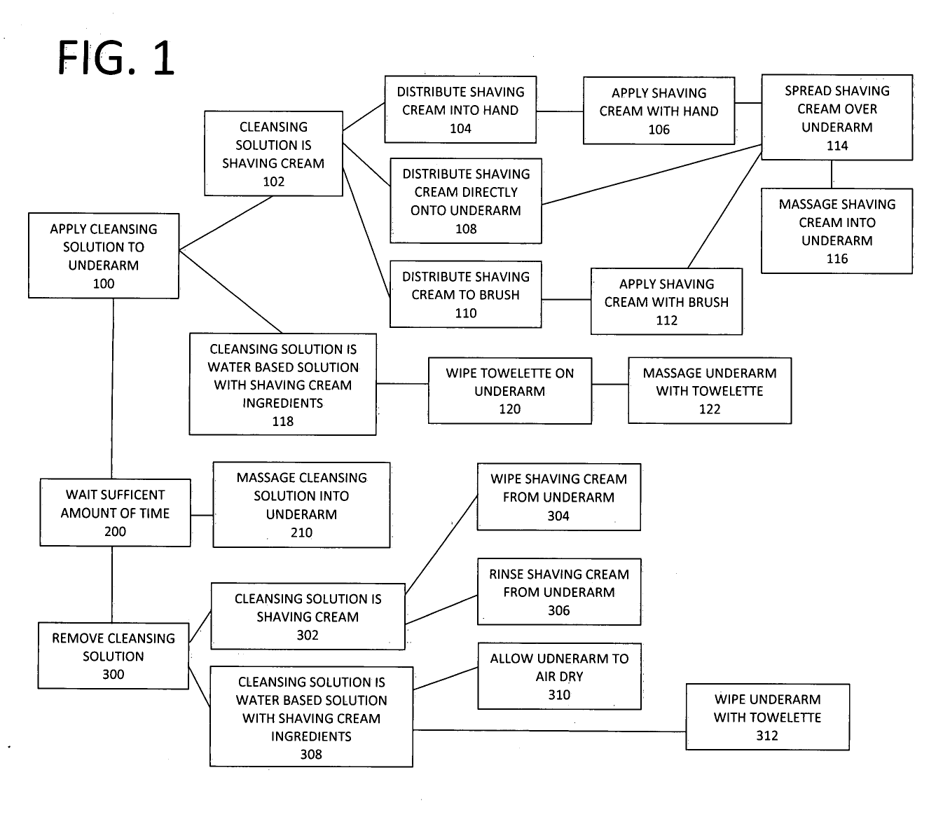

Referring to FIG. 1, a block diagram showing the steps of method 10 of the present invention is provided. The basic steps of method 10 include: applying the cleansing solution to the underarm 100; waiting a sufficient amount of time 200 for the cleansing solution to cleanse the underarm; and removing the cleansing solution from the underarm 300.

### Applying the Cleansing Solution to the Underarm 100

Steps 102-116 show preferred embodiments in which the cleansing solution is shaving cream 102. Most shaving creams are water based solutions containing at least a combination of surfactant and detergent ingredients, along with emulsifier and propellant ingredients. The ingredients of the shaving cream sold under the registered trademark BARBASOL BEARD BUSTER, which is the preferred shaving cream of the method of the present invention, has the following ingredients: water, stearic acid, triethanolamine, isobutane, laureth-23, fragrance, propane, and sodium lauryl sulfate. Triethanolamine is an emulsifier. Isobutane is a propellant. Laureth-23 is a detergent, surfactant, and foaming agent. Sodium lauryl sulfate is a surfactant with detergent properties. It is noted that, although the shaving cream sold under the registered trademark BARBASOL BEARD BUSTER is the preferred shaving cream of the method of the present invention, other brands of shaving cream containing laureth-23 and sodium lauryl sulfate may be substituted to achieve similar results. As explained above, not all shaving creams containing laureth-23 and sodium lauryl sulfate are preferred, however. There are three preferred embodiments for applying the cleansing solution to the underarm 100 when the cleansing solution is shaving cream 102 (hereinafter “applying the shaving cream to the underarm 102”). In other embodiments, any means commonly used in the art of applying shaving cream may be substituted, however. In the first preferred embodiment, the step of applying the shaving cream to the underarm 102 entails the steps of distributing the shaving cream into the hand 104 and applying the shaving cream to the underarm with the hand 106. Distributing the shaving cream into the hand 104 may be by any means commonly used in the art. If the shaving cream is condensed and housed in a canister, for example, the distribution is by spraying. If the shaving cream is housed in a tin, for another example, the distribution is by dipping the fingers or a brush into the tin to remove an amount of the shaving cream.

In the second preferred embodiment, the step of applying the shaving cream to the underarm 102 includes the step of distributing the shaving cream directly onto the underarm 108. This step is used in embodiments of the method where condensed shaving cream from a canister is used, for example. Under such circumstances, the shaving cream is sprayed directly onto the underarm.

In the third preferred embodiment, the step of applying the shaving cream to the underarm 102 includes the steps of distributing the shaving cream to a brush 110 and applying the shaving cream to the underarm with the brush 112. The distribution of the shaving cream may be by any means commonly used in the art, as discussed above, with reference to step 104. In some embodiments, the shaving cream dispenser has a built-in brush onto which the shaving cream can be distributed. In other embodiments, the brush has a separate implement from the shaving cream dispenser, onto which the shaving cream can be distributed.

In each of these preferred embodiments, the step of applying the shaving cream to the underarm 102 includes the steps of spreading the shaving cream over the underarm 114 and massaging the shaving cream into the underarm 116. The step of spreading the shaving cream over the underarm 114 involves ensuring that the entire underarm area is covered with shaving cream. The step of massaging the shaving cream into the underarm 116 involves ensuring that the shaving cream comes into contact with every surface area of the underarm, including hair shafts, outer hair follicles, and the skin between hairs.

The amount of shaving cream to be applied in step 102 varies depending on several factors, but in all cases is no less than a small approximately golf ball sized dollop of shaving cream that can be held by only the tips of the middle three fingers. Factors that may affect the amount of shaving cream needed include the area of the underarm to be cleansed, the amount of hair present in the underarm to be cleansed, the amount of time passed since the underarm to be cleansed has been washed, the gender and age of the owner of the underarm to be cleansed, and the personal preference of the performer of method 10 of the present invention.

Steps 118-122 show the preferred embodiment when the cleansing solution is a fluid solution with shaving cream ingredients 118, such as surfactants and detergents. When the cleansing solution is a fluid solution with shaving cream ingredients 118, the preferred cleansing solution is a solution containing laureth-23, sodium lauryl sulfate, and fragrance. In the preferred method for applying the cleansing solution to the underarm 100 when the cleansing solution is a fluid solution with shaving cream ingredients 118 (hereinafter “applying the fluid solution to the under arm 118”), a towelette is impregnated with the solution. Thus the preferred method of performing step 118 includes the step of wiping the towelette on the underarm 120. This preferably also includes the step of massaging the underarm with the towelette 122, to ensure that every surface of the underarm is exposed to the solution.

### Waiting a Sufficient Amount of Time 200

This step entails waiting a sufficient amount of time for the cleansing solution to counteract the bacteria causing underarm malodor. This amount of time may be as little as the few seconds it may take for the step of applying the cleansing solution to the underarm 100, and may be up to two minutes, however. The amount of time is preferably thirty to sixty seconds, however. The performer of the method preferably continues the step of massaging the cleansing solution into the underarm 210, whether this is a continuation of step 116 or 122, while he is performing the step of waiting a sufficient amount of time 200.

This step is necessary to allow the cleansing solution to cleanse the underarm and reduce or eliminate its malodor. When the cleansing solution is shaving cream 102, the ingredients of laureth-23 and sodium lauryl sulfate are the ingredients that cleanse the underarm. Laureth-23 is a polyethylene glycol of lauryl alcohol that acts as a surfactant, deodorant, cleansing agent, and emulsifier. Sodium lauryl sulfate is also a surfactant that has detergent properties and is often used in the removal of oily stains and residues. Both of these ingredients cleanse the underarm. Shaving cream also contains one or more fragrance ingredients. These do not cleanse the underarm, but mask or reduce malodor. It is these ingredients that are included in the cleansing solution when the cleansing solution is a fluid solution with shaving cream ingredients 118. The step of waiting a sufficient amount of time 200 allows these ingredients time to act on the underarm.

Removing the Cleansing Solution from the Underarm 300

This step removes the cleansing solution from the underarm without removing the hair therefrom. Presumably, this step also removes bacteria and other odor-causing agents with the cleansing solution. Removing the cleansing solution from the underarm 300 when the cleansing solution is shaving cream 302 preferably entails wiping the shaving cream from the underarm 304 and/or rinsing the shaving cream from the underarm 306 with water. The step of wiping the shaving cream from the underarm 304 is preferably performed by wiping the shaving cream away with a towelette, which may be impregnated with water, but may be performed by any means commonly used for such a task, such as with a hand or washcloth. This step 304 may be executed alone or in conjunction with rinsing the shaving cream from the underarm 306. Step 306 may also be executed alone.

As discussed above, when the cleansing solution is a fluid solution with shaving cream ingredients 118/308, in the preferred embodiment, a towelette is impregnated with the solution. Removing the cleansing solution 300 when the cleansing solution is a fluid solution with shaving cream ingredients 308 is preferably achieved by allowing the underarm to air-dry 310 or by wiping the underarm with a towelette 312.

Steps 304, 310, and 312 are particularly useful when the performer of method 10 does not desire or does not have the time or ability to take a full shower. Eliminating a shower from method 10 requires only that the person whose underarms are being cleansed open, partially remove, or remove his shirt, which may be done even in a public place, such as a public rest room. He may quickly and easily refresh his underarms wherever he is.

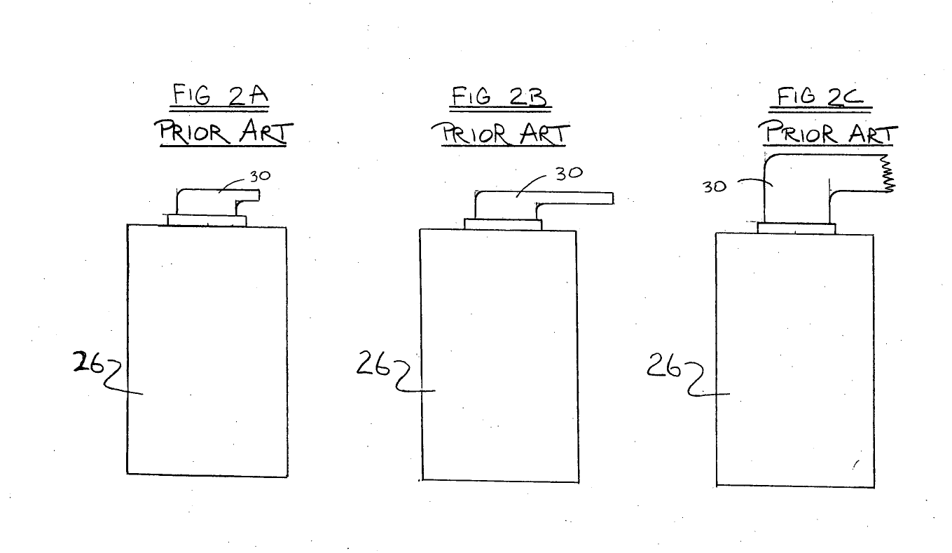

Shaving creams come in a variety of dispensers. In modern times, most of these dispensers are cans or canisters that house the shaving cream under pressure. In addition to such standard cans, some shaving cream dispensers, such as that taught in U.S. Pat. No. 4,974,984, include a brush top that aid in applying the shaving cream once dispensed. Others, such as that taught in U.S. Pat. No. 5,415,488, include other attachments that extend away from the canister to aid in dispensing the shaving cream directly on the surface to be shaved. FIGS. 2A-C depict several prior art shaving cream dispensers. FIG. 2A shows a standard canister. FIG. 2B shows a standard canister with an extended nozzle for applying the shaving cream directly on the surface to be shaved. FIG. 2C shows a canister with a brush head to aid in applying and distributing the shaving cream. FIGS. 2A-2C each include a shaving cream dispenser 26, which includes a nozzle 30, each nozzle 30 being different as described above.

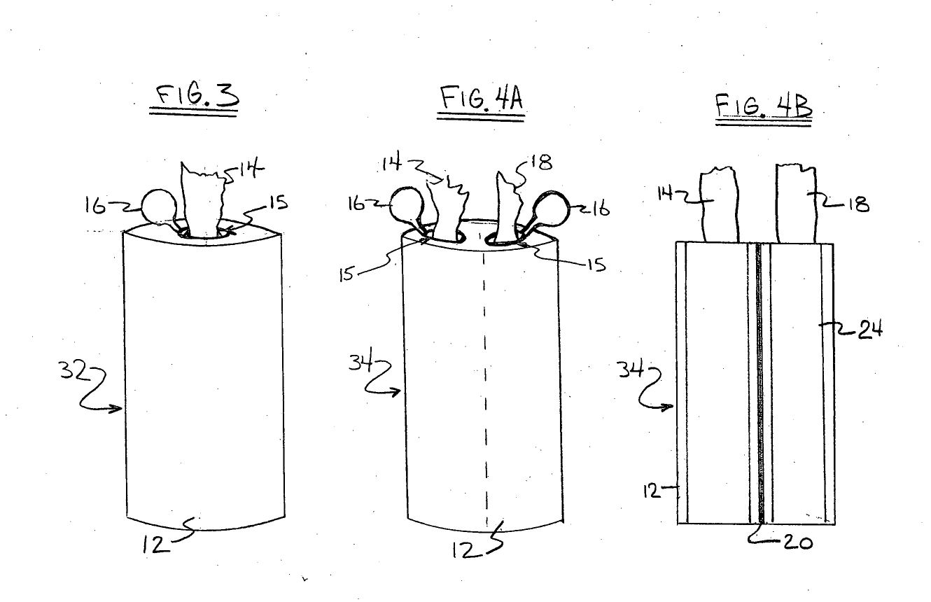

Referring now to FIG. 3 a preferred embodiment of the kit of the present invention is shown. Kit 32 includes towelette dispenser 12, hole 15, cleansing solution towelette 14, and dispenser cap 16. Towelette dispenser 12 is preferably plastic, cylindrical, and shaped and dimensioned to be easily held in an average human hand. Towelette dispenser 12 is dimensioned and arranged to house and dispense cleansing solution towelettes 14. Towelette dispenser 12 includes hole 15, out of which cleansing solution towelette 14 may be pulled free of towelette dispenser 12. Hole 15 is small enough so that there is some resistance when cleansing solution towelette 14 is pulled from towelette dispenser 12. Towelette dispenser 12 also includes dispenser cap 16, which is attached to towelette dispenser 12, and may be removably affixed over hole 15 when no cleansing solution towelette 14 is being removed from towelette dispenser 12.

Cleansing solution towelette 14 is a disposable towelette impregnated with a fluid cleansing solution containing surfactant and detergent ingredients commonly found in shaving creams. A preferred cleansing solution, for example, is a solution containing laureth-23, sodium lauryl sulfate, and fragrance. Although only part of one cleansing solution towelette 14 is shown coming out of hole 15, it is understood that a plurality of cleansing solution towelettes 14 are present in a roll 22 (see FIG. 4B) within the body of towelette dispenser 12.

Referring now to FIGS. 4A and 4B, another preferred embodiment of the kit of the present invention is shown. Common reference numbers in other figures refer to common features of the present invention. Kit 34 includes all of the features of kit 32, but also includes removal towelettes 18 and barrier 20. Removal towelette 18 is a towelette that is either dry or is impregnated with water. FIG. 4B shows a cross-section of towelette dispenser 12. In this view, we see rolls 22, 24 of cleansing solution towelettes and removal towelettes 14, 18, respectively, and barrier 20 between them. Barrier 20 partitions towelette dispenser 12 so that cleansing solution towelette roll 22 and removal towelette roll 24 are not in contact and the cleansing solution does not infiltrate the removal towelette roll 24.

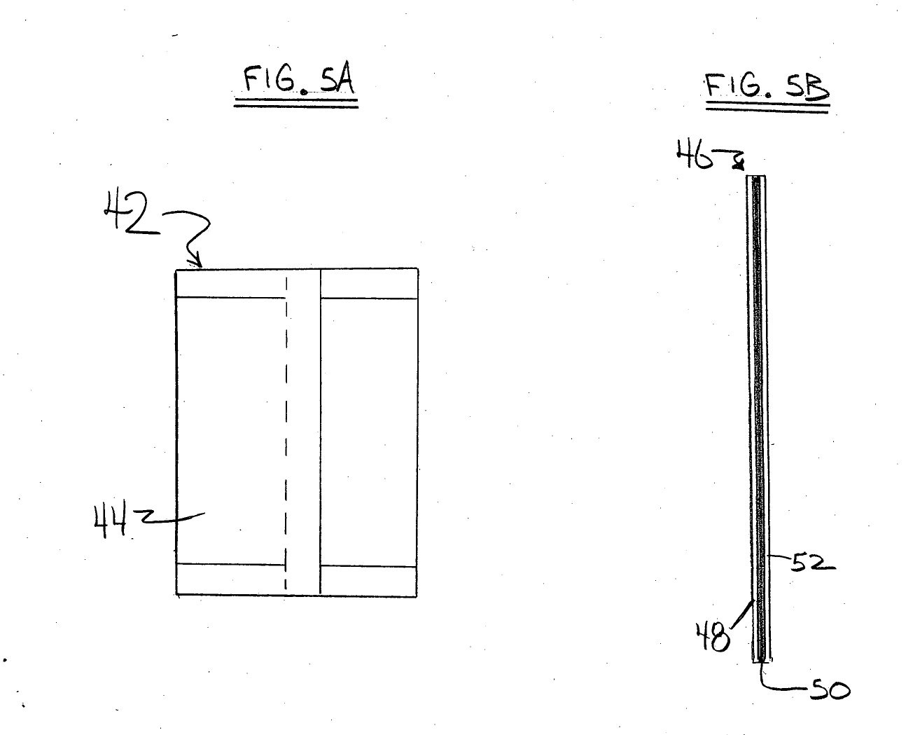

Referring now to FIGS. 5A and 5B, another preferred embodiment of the kit of the present invention is shown. Kit 42 includes wrapper 44. In this embodiment, each towelette is individually wrapped for easy transport. Wrapper 44 is dimensioned and arranged to house cleansing solution towelette 14, and is sealed to be liquid-tight so that the fluid solution cannot evaporate before cleansing solution towelette 14 is used. Wrapper 44 is preferably made of a flexible plastic or metallic material. Wrapper 44 may be easily opened to reveal the still moist cleansing solution towelette 14 within.

In a preferred embodiment, wrapper 44 houses not cleansing solution towelette 14, but combination towelette 46, shown in FIG. 5B. Combination towelette 46 is two-sided. On one side 48, combination towelette 46 is impregnated with the cleansing solution. On the other side 52, combination towelette 46 is dry or is impregnated with water. Sides 48, 52 are separated by barrier 50 so that the cleansing solution does not infiltrate the side used for removal of the cleansing solution. Barrier 50 is preferably made of a flexible, non-diffusive plastic or metallic material.

Kits 32, 34, and 42 can be used to quickly and easily cleanse one's underarms without needing a water source. In preferred embodiments, they are small enough to fit in a purse, briefcase, or backpack, so that they can be taken anywhere. A user need only wipe his underarm with cleansing solution towelette 14 or side 48 of combination towelette 46 to cleanse and refresh his underarms. With kit 34 or kit 42 containing combination towelette 46, he may also rinse the cleansing solution from his underarms with removal towelette 18 or side 52 of combination towelette 46. It is possible that the user could use kits 32, 34, 42 without even removing his shirt. In this way, or by opening, partially removing, or removing his shirt, he may quickly and easily cleanse and refresh his underarms, even in a public place, such as a public restroom.

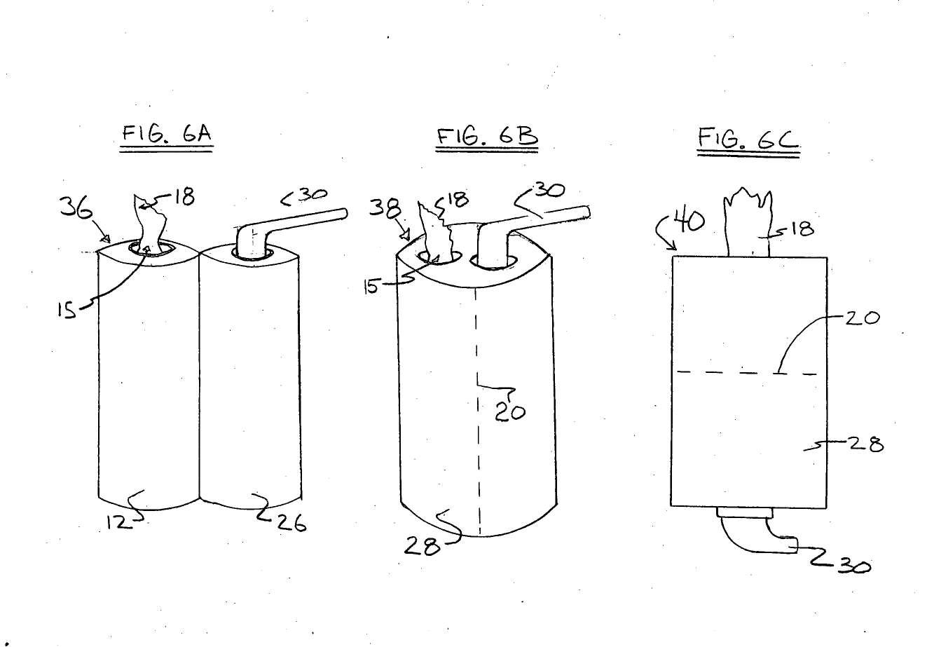

Referring now to FIG. 6A, another preferred embodiment of the kit of the present invention is shown. Kit 36 includes towelette dispenser 12 and shaving cream dispenser 26. Towelette dispenser 12 and shaving cream dispenser 26 are separate containers, but attached to one another by common means in the art, such as by adhesives. Towelette dispenser 12 houses and dispenses removal towelettes 18, as described above with reference to FIGS. 4A and 4B. Shaving cream dispenser 26 is dimensioned and arranged to house and dispense shaving cream. Shaving cream dispenser 26 includes nozzle 30 through which shaving cream housed within shaving cream dispenser 26 flows.

Referring now to FIGS. 6B and 6C, preferred embodiments, kits 38, 40, respectively, of the present invention are shown. Each of kit 38, 40 include combined dispenser 28. Combined dispenser 28 includes both removal towelettes 18 and shaving cream. Shaving cream is dispensed from combined dispenser 28 through nozzle 30. Shaving cream and removal towelettes 18 are separated and kept from contact by barrier 20 (shown in a dotted line as being within combined dispenser 28), as described above with reference to FIGS. 4A and 4B. In kit 38, the partitions are aligned side by side. In kit 40, the partitions are aligned one on top of the other.

Kits 36, 38, and 40 can also be used to quickly and easily cleanse one's underarms. In preferred embodiments, these kits are also small enough to be carried in a purse, briefcase, or backpack, so can be taken anywhere. The user need only dispense a quantity of shaving cream from nozzle 30 and apply it to his underarms. He can then wipe the shaving cream away with removal towelettes 18. He is left with clean, fresh underarms. These kits may be used anywhere to easily and quickly cleanse underarms.

Although the present invention has been described in considerable detail with reference to certain preferred versions thereof, other versions would be readily apparent to those of ordinary skill in the art. Therefore, the spirit and scope of the description should not be limited to the description of the preferred versions contained herein.

What is claimed is:

1. A method for cleansing underarms comprising the steps of:
applying an amount of shaving cream comprising laureth-23 and sodium lauryl sulfate to a malodorous underarm;
waiting a sufficient amount of time; and
removing said shaving cream from said underarm without removing hair therefrom.

2. The method as claimed in claim 1, wherein said shaving cream further comprises the ingredients of water; stearic acid; triethanolamine; isobutane; at least one fragrance; and at least one propellant.

3. The method as claimed in claim 1, wherein said step of applying an amount of shaving cream comprises the steps of:
distributing said amount of shaving cream into a hand of a performer of said method;
applying said amount of shaving cream to said underarm with said hand;
spreading said shaving cream over said underarm; and
massaging said shaving cream into said underarm.

4. The method as claimed in claim 1, wherein said step of applying an amount of shaving cream comprises the steps of:
distributing said amount of shaving cream directly onto said underarm;
spreading said shaving cream over said underarm; and
massaging said shaving cream into said underarm.

5. The method as claimed in claim 1, wherein said step of applying an amount of shaving cream comprises the steps of:
distributing said amount of shaving cream onto a brush;
applying said amount of shaving cream to said underarm with said brush;
spreading said shaving cream over said underarm; and
massaging said shaving cream into said underarm.

6. The method as claimed in claim 1, further comprising the step of massaging said shaving cream into said underarm.

7. The method as claimed in claim 1, wherein said step of removing said shaving cream from said underarm comprises the step of wiping said shaving cream from said underarm.

8. A method for cleansing underarms comprising the steps of:
wiping a towelette over a malodorous armpit, wherein said towelette is impregnated with a cleansing solution comprising laureth-23 and sodium lauryl sulfate;
waiting a sufficient amount of time; and
removing said cleansing solution from said underarm.

9. The method as claimed in claim 8, further comprising the step of massaging said underarm with said towelette.

10. The method as claimed in claim 8, wherein said step of removing said cleansing solution comprises allowing said underarm to air-dry.

11. The method as claimed in claim 8, wherein said step of removing said cleansing solution comprises wiping said underarm with a towelette impregnated with water.

12. A kit for cleansing underarms comprising:
a towelette dispenser; and
at least one towelette disposed within said towelette dispenser, wherein at least one of said at least one towelette is impregnated with a cleansing solution comprising laureth-23 and sodium lauryl sulfate.

13. The kit as claimed in claim 12, wherein said at least one towelette comprises a plurality of cleansing towelettes arranged in a roll and wherein said towelette dispenser comprises a cylindrical housing having a removable top.

14. The kit as claimed in claim 13, further comprising a plurality of removal towelettes arranged in a roll;
wherein said towelette dispenser is further dimensioned and arranged to house and dispense said removal towelettes;
wherein said towelette dispenser further comprises a barrier between said cleansing towelettes and said removal towelettes such that said cleansing towelettes and said removal towelettes are not in contact and such that said cleansing solution impregnated within said cleansing towelettes does not contact said removal towelettes.

15. The kit as claimed in claim 12, wherein said towelette dispenser comprises an airtight wrapper, wherein said at least one towelette comprises a cleansing towelette and a removal towelette, and wherein said kit further comprises a barrier between said cleansing towelette and said removal towelette such that said cleansing towelettes and said removal towelettes are not in contact and such that said cleansing solution impregnated within said cleansing towelette does not contact said removal towelette.
